(19) United States
(12) Patent Application Publication
(10) Pub. No.: US 2003/0118577 A1
(43) Pub. Date: Jun. 26, 2003

(54) Use of analogues of superoxide dismutase for treating hepatocellular insufficiencies

(76) Inventors: Bernard Weill, Eaubonne (FR); Frederic Batteux, Paris (FR); Pierre-Jacques Ferret, Boulogne (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/240,270

(22) PCT Filed: Mar. 28, 2001
(86) PCT No.: PCT/FR01/00939

(30) Foreign Application Priority Data
Mar. 28, 2000 (FR) 00/03887

(51) Int. Cl. A61K 38/44; A61K 31/555

(52) U.S. Cl. 424/94.4; 514/184

(57) ABSTRACT

The invention concerns the use of a compound having a superoxide dismustase activity, in particular a non-peptide analogue of the superoxide dismutase to obtain a medicine for preventive or curative treatment of hepatocellular insufficiency. Said medicine can be used in particular for treating hepatocellular insufficiencies of toxic or viral origin.

The invention relates to novel uses of superoxide dismutase mimetics in the context of the treatment of hepatocellular insufficiency.

The term hepatocellular insufficiency denotes a combination of pathological manifestations resulting from the destruction of the hepatocytes. Depending on the extent of the cellular destruction, these clinical manifestations are more or less serious and reversible. In extreme cases, the massive and sudden destruction of the hepatocytes leads to acute hepatic insufficiency, also called fulminant hepatitis, which can cause death within a few days.

Among the most frequent causes of destruction of hepatocytes which can lead to hepatocellular insufficiency, there may be mentioned in particular viral infections, due to various types of hepatitis virus, and intoxications, in particular by certain medicaments or by alcohol.

Several experimental animal models of hepatocellular insufficiency of various origins are currently available which make it possible in particular to experimentally induce acute hepatic insufficiency, and to study the mechanisms leading to cellular destruction. Depending on the initial factor responsible for cellular destruction, various mechanisms have thus been proposed.

For example, acute hepatic insufficiency of toxic origin, induced in particular by acetaminophen, results from saturation of the normal hepatic detoxification mechanisms. Indeed, at pharmacological doses, acetaminophen is mainly eliminated by glucoro- and sulfoconjugation, but is also oxidized by cytochrome P450 to N-acetyl-p-benziquinone-imine (NAPQI), which can normally then be eliminated after conjugation with glutathione. In the event of overdosage, the saturation of the glucoro- and sulfoconjugation pathways and an increased production of NAPQI [PRESCOTT, Drugs, 25, 290-314, (1983)] are observed. This very reactive metabolite is supposed to be the main effector of lesions of hepatocytes, and the drug treatments proposed are essentially based on the use of antioxidants such as N-acetyl-L-cysteine whose role is to allow the reconstitution of the intracellular glutathione reserves and the neutralization of NAPQI. The efficacy of these treatments is however not constant [CARACENI and VAN THIEL, Lancet, 345, 163-169, (1995); SCHIODT et al., N. Engl. J. Med., 337, 1112-1117, (1997)] and, in the case of fulminant hepatitis, liver transplant currently constitutes the only really effective treatment.

The mechanism proposed in the case of hepatocellular insufficiency of viral origin is different; it involves mainly apoptosis induced by the interaction between the Fas receptor and its ligand (Fas/FasL). Under physiological conditions, the induction of Fas/FasL apoptosis participates in anti-viral defence, by allowing the destruction, by the cytotoxic T lymphocytes, of infected cells expressing the viral antigen. An exaggerated amplification of this phenomenon causes massive destruction of hepatocytes, which, if it occurs suddenly, can take the form of fulminant hepatitis.

In the case of hepatocellular insufficiency induced by alcohol, several mechanisms have been proposed which involve in particular apoptosis mediated by death receptors, and the generation of toxic metabolites such as acetaldehyde.

The inventors have now observed that manganese superoxide dismutase (Mn-SOD) mimetics very effectively protect the hepatocytes from the destructive effects of toxic substances or of viral infections, and thus possess both a preventive and curative effect on hepatocellular insufficiency, whether it is acute or chronic, and whether it is of viral or toxic origin (drug origin or induced by alcohol).

The term superoxide dismutase (SOD) denotes a family of metalloproteins (EC 1.15.1.1) whose activity occurs in the detoxification of free oxygenated radicals, by catalyzing the dismutation of the superoxide anion (O.⁻²) to hydrogen peroxide (H₂O₂). Various types of superoxide dismutase can be distinguished, among which there may be mentioned in particular copper-zinc superoxide dismutases (CuZnSOD), also known by the name superoxide dismutase-1, which, in eukaryotic organisms, are mainly located in the cytoplasm, and manganese superoxide dismutases (MnSOD), also known by the name superoxide dismutase-2, which are found mainly in prokaryotes and in the intracellular organelles of eukaryotic cells.

The hydrogen peroxide, generated by the SOD activity, which is itself toxic and can generate other free oxygenated radicals, is then detoxified by means of various enzymes, in particular catalase, glutathione peroxidase, and thioredoxin.

The role played by the free oxygenated radicals in the appearance and the development of cancers as well as numerous inflammatory and autoimmune diseases is now recognized. It has therefore been proposed to use products possessing an SOD activity for the treatment of these pathologies. As the administration of SOD poses certain problems (in particular of bioavailability, and of side effects resulting from the immunogenicity of the molecule), these products are most often nonpeptide SOD mimetics.

The use of SOD mimetics has thus been proposed in the context of various pathologies involving the superoxide anion or its metabolites [for a review cf. PATEL and DAY, Trends Pharmacol. Sci., 20, 359-364 (1999)]. Their potential therapeutic efficacy in vivo has up until now been studied mainly on animal models of post-ischemia reperfusion injury, or of inflammation induced by the superoxide anion produced by polynuclear neutrophiles, [cf. for example SALVEMINI et al.: Br. J. Pharmacol., 127, 685-692, (1999); Science, 286, 304-305, (1999)].

In the case of hepatic pathologies, the few experiments reported have related to CuZnSOD or its mimetics. Application EP 0333490 thus reports that hepatic impairments induced by the administration of acetaminophen or of galactosamine are reduced by the simultaneous administration of CuZnSOD. MIESEL et al. (Gen. Pharmac., 26, 1261-1266, 1995) reports that the hepatic impairments resulting from the administration of galactosamine-LPS are reduced by the prior administration of CuPu(Py)2, which is a nonpeptide CuZnSOD mimetic.

The inventors have, in a first instance, sought the effect of SOD mimetics on acute hepatic insufficiency of toxic origin, by using an experimental model of acute hepatic insufficiency induced by the administration of acetaminophen. They observed that the administration of an MnSOD mimetic, MnTBAP, makes it possible to very significantly increase the survival rate after administration of a lethal dose of acetaminophen, and to considerably reduce the toxic effects thereof, whereas, under the same conditions, the administration of a CuZnSOD mimetic has only little or no effect.

In addition, they observed that the beneficial effects of an MnSOD mimetic are observed not only when the latter is administered preventively, but also when it is administered curatively, that is to say after the appearance of the first hepatotoxic effects.

The inventors investigated whether a similar effect was observed on hepatocellular insufficiency of viral origin, using an experimental model of fulminant hepatitis resulting from Fas-dependant apoptosis induced by the administration of anti-Fas antibody possessing a similar activity to that of FasL. They observed, very surprisingly, that the beneficial effects of an MnSOD mimetic on the reduction of the hepatic lesions and the survival rate of the animals are even greater than in the case of acute hepatic insufficiency of toxic origin.

The inventors also investigated the effects of MnSOD mimetics on hepatocellular insufficiency of toxic origin which can be induced in particular by alcohol, using an experimental model of intoxication with dimethylnitrosamine (DMNA) administered at the dose of 10 mg/kg in mice. This model is similar to intoxication with alcohol because the lesions induced by DMNA and alcohol are identical.

They observed, on histological sections made at different intoxication times, that MnTBAP delays the appearance of hepatic lesions induced by DMNA.

The subject of the present invention is the use of an MnSOD mimetic for producing a medicament intended for the preventive or curative treatment of hepatocellular insufficiency.

MnSOD mimetics which can be used in the context of the present invention are known per se. SOD mimetics are generally nitrogen-containing macrocyclic derivatives chelating a metal, which is manganese in the case of MnSOD mimetics. Among those whose SOD mimetic activity has been best characterized in vivo, there may be mentioned in particular metalloporphyrin derivatives [PASTERNACK et al., Inorg. Biochem., 15, 261-267 (1981)], such as MnTBAP [Mn(III) tetrakis(5,10,15,20-benzoic acid)porphyrin], or macrocyclic derivatives such as those described in U.S. Pat. No. 5,874,421 in the name of RILEY et al., or in the publication by WEISS et al., [J. Biol. Chem, 271, 26149-26156 (1996)], who propose their use in the treatment of pathologies resulting from the toxic effects of free oxygenated radicals.

In the context of the implementation of the present invention, it may be advantageous, in some cases, to use an SOD mimetic also possessing one or more other activities involved in the detoxification of reactive oxygenated species other than the superoxide anion. By way of example, there may be mentioned MnTBAP; this compound is known to possess a catalase activity in addition to its SOD activity. Furthermore, the inventors observed that it possessed, in addition, a glutathione peroxidase activity, which participates, like catalase activity, in the detoxification of hydrogen peroxide, which may be advantageous, for example, in the context of the treatment of acute hepatic insufficiency of toxic origin.

According to a preferred embodiment of the present invention, said MnSOD mimetic is used for producing a medicament intended for the preventive or curative treatment of hepatocellular insufficiency of toxic origin, and in particular for the treatment of hepatocellular insufficiency induced by acetaminophen, or of hepatocellular insufficiency induced by alcohol.

According to another preferred embodiment of the present invention, said MnSOD mimetic is used for producing a medicament intended for the preventive or curative treatment of hepatocellular insufficiency resulting from Fas-dependent apoptosis, apoptosis of the hepatocytes mediated by death receptors, and in particular for the treatment of hepatocellular insufficiency of viral origin.

In a particularly advantageous manner, said MnSOD mimetic may be used for producing a medicament intended for the treatment of acute hepatocellular insufficiency manifesting itself in particular in the form of fulminant hepatitis.

The use, in accordance with the invention, of an MnSOD mimetic makes it possible, in addition, because of the protection provided with respect to tissue lesions resulting from the destruction of the hepatocytes, to prevent the constitution of fibrotic lesions which can result from the cicatrization of these lesions.

Said MnSOD mimetic may also be used for protecting hepatic grafts during their preservation, in order to prevent hepatocellular lesions resulting from ischemia of the hepatic grafts after their removal and during their preservation.

For the implementation of the present invention, the MnSOD mimetics may be used in the customary formulations and routes of administration for these types of compound, such as those described for example in U.S. Pat. No. 5,874,421.

Advantageously, they will be used in the context of formulations allowing the administration of a dose of active ingredient of between 0.1 and 10 mg/kg/day (preventive administration) or of between 5 and 50 mg/kg/day (curative administration); it is clearly understood however that a person skilled in the art can adapt these doses in particular according to the patients' age, weight and pathology.

These compounds may be administered by the oral route, by inhalation, by the rectal route, by the cutaneous route or by the general route, in particular by subcutaneous, intramuscular or intravenous injections, according to the desired formulation or galenic form. Other routes of administration may be envisaged if they increase the efficacy, the bioavailability or the tolerance of the products.

In the case of a use ex vivo on a hepatic graft, said MnSOD mimetic may be added to the perfusion fluid and/or to the preservation fluid for said graft.

The present invention will be understood more clearly with the aid of the additional description which follows, which refers to nonlimiting examples demonstrating the activity of an MnSOD mimetic on hepatocellular insufficiency of various origins.

### EXAMPLE 1

### Activity of MnTBAP on Acute Hepatic Insufficiency Induced by Acetaminophen

The intraperitoneal injection of acetaminophen into mice induces a severe hepatotoxicity, the degree of which may be evaluated by the survival of the animals, measuring transaminase activities, and macroscopic and microscopic examination of the livers.

Survival of the Animals

The MnTBAP (marketed by ALEXIS BIOCHEMICALS) is administered in the form of a bolus, by the intraperitoneal route.

The acetaminophen in solution at 100 mg/ml in PBS at pH 7.4 is administered by the intraperitoneal route.

In a first series of experiments, a group of mice received a dose of 1000 mg/kg of acetaminophen; a second group received a dose of 1000 mg/kg of acetaminophen and a dose of 10 mg/kg of MnTBAP administered either 2 h before the acetaminophen or 6 h after; a control group received either MnTBAP alone (10 mg/kg), or PBS alone.

The survival of the animals is monitored for 24 hours after the administration of acetaminophen.

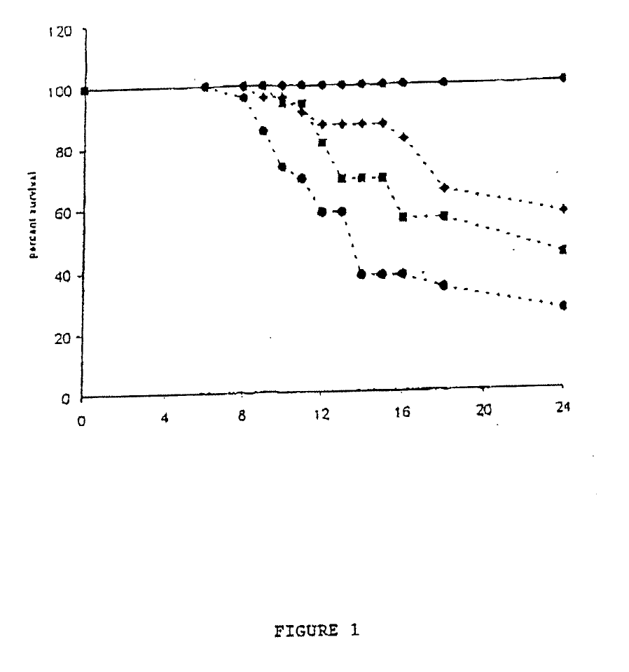

The results are illustrated by FIG. 1, which represents the percentage survival as a function of time. These results show that 24 hours after the injection of acetaminophen, 74% of the animals which did not receive MnTBAP (●) died, whereas the rate of survival is greater than 40% in the animals which received MnTBAP after the administration of acetaminophen (▪), and of the order of 60% in the animals which received MnTBAP before the administration of acetaminophen (♦). No death is observed in the animals which received MnTBAP or PBS alone.

Assay of Transaminases

In a first series of experiments, a group of mice received a dose of 1000 mg/kg of acetaminophen; a second group received the same dose of acetaminophen, and a dose of 10 mg/kg of MnTBAP administered 2 h before the acetaminophen; a control group received either MnTBAP alone (10 mg/kg), or PBS alone.

The serum transaminases ALAT and ASAT are assayed 12 hours and 24 hours after the administration of acetaminophen.

Since the results risked being biased by the fact that the mice surviving 24 hours after the administration of acetaminophen were probably, regardless of the group involved, those which had the lowest transaminase activity, a second series of experiments were carried out, administering 500 mg/kg of acetaminophen, the other experimental conditions remaining the same. At this dosage, all the mice were still alive after 24 hours.

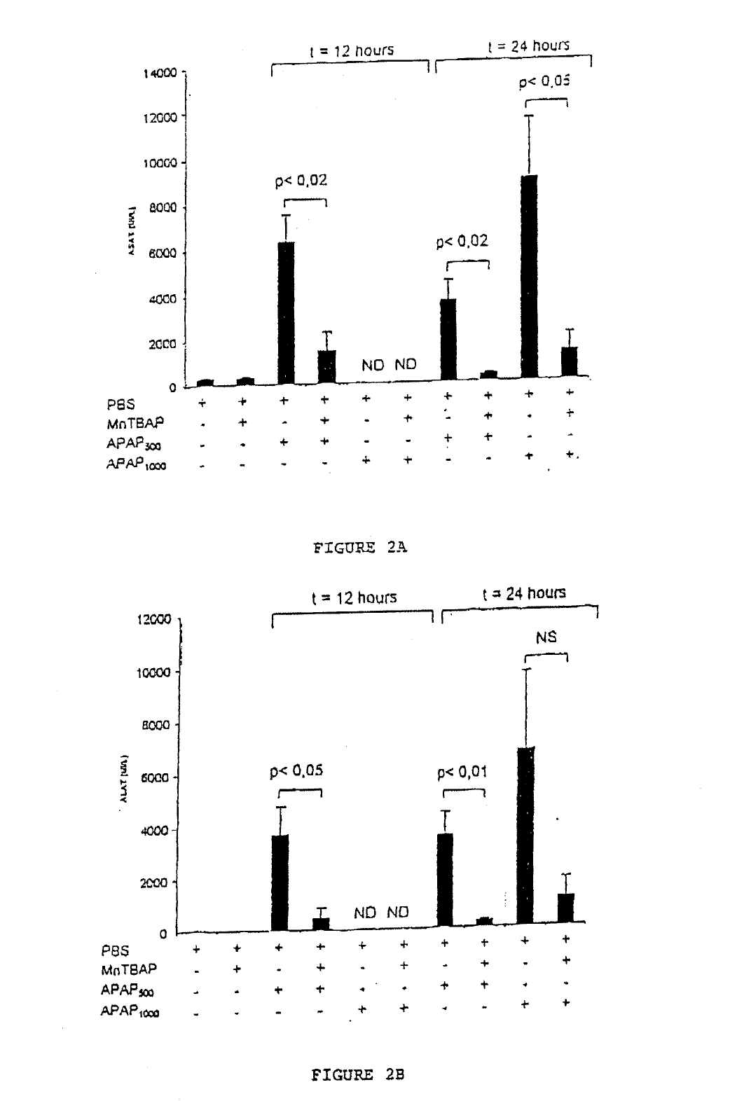

The results are illustrated by FIGS. 2A and 2B, which represent the ASAT and ALAT activity, respectively, according to the products administered.

Among the mice which received 1000 mg/kg of acetaminophen (APAP₁₀₀₀), 6-fold lower transaminase activities were observed, after 24 hours, in those which received beforehand a treatment with MnTBAP. Among the mice which received 500 mg/kg of acetaminophen (APAP₅₀₀), 10-fold lower transaminase activities were observed, after 24 hours, in those which received beforehand a treatment with MnTBAP.

These results show that, in all cases, the administration of MnTBAP reduces the transaminase activities, which reflect hepatic cytolysis.

Histological Study

In each of the groups, the livers of several animals were removed, in order to carry out a histological study. In the case of mice which received acetaminophen, a lot fewer apoptotic lesions are observed in the animals treated with MnTBAP than in the untreated animals. No apoptotic lesion is visible in the mice of the control group which did not receive acetaminophen.

### EXAMPLE 2

### Activity of MnTBAP on Acute Hepatic Insufficiency Induced by Anti-Fas Antibodies

Anti-Fas antibodies possessing a similar activity to that of FasL are used in experimental models of apoptosis. The injection of these antibodies into mice causes fulminant hepatitis due to a massive apoptosis of the hepatocytes, causing the death of the mice within a few hours following the injection [OGASAWARA et al., Nature, 364, pp. 806-809, (1993); NAGATA, Prog. Mol. Subcell. Biol., 16, pp. 87-103, (1996)].

27 control mice received, by intravenous injection, 6 μg of an anti-Fas monoclonal antibody (clone J02; PHARMINGEN) diluted in 100 μl of physiological saline; a second group of 15 mice received the same treatment, preceded by the administration, 2 hours beforehand, of 10 mg/kg of MnTBAP, as described in Example 1 above; a control group received MnTBAP alone (10 mg/kg).

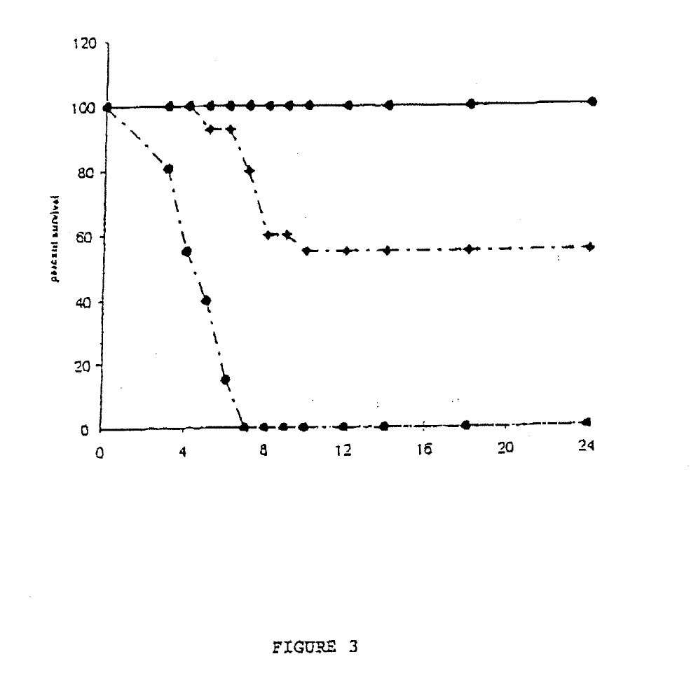

The results are illustrated by FIG. 3, which represents the percentage survival as a function of time. These results show that 7 hours after the injection of anti-Fas antibody, all the mice which did not receive MnTBAP (●) died, whereas the rate of survival at 24 hours is of the order of 60% in the animals which received MnTBAP beforehand (♦).

### EXAMPLE 3

### Activity of MnTBAP on Hepatocellular Inusfficiency Induced by Chronic Intoxication with DMNA.

Intraperitoneal administration of 10 mg/kg/day of DMNA three times per week induces, in dogs and rodents, centrolobular and periportal lesions comprising fibrosis at the 4^{th} week, and cirrhosis at the 13^{th} week [RISTELLI et al., J. Biochem., 158, 361-367, (1976); MADDEN et al., Surgery, 68, 260-267, (1970)]. The administration of MnTBAP by the intraperitoneal route at the dose of 10 mg/kg, 24 hours after each administration of DMNA, prevents the constitution of objectively viable fibrosis on histological examination after HES, GORDON, MASSON and PAS staining.

### EXAMPLE 4

### Activity of MnTBAP on Ischemic Lesions of Hepatic Grafts

Ischemia of hepatic grafts after their removal and during their preservation at 4° C. causes hepatocytic lesions.

The intensity of the lesions may be evaluated on the concentration of transaminases in the preservation fluid.

Mouse livers were surgically removed, rinsed with Belzer's preservation fluid supplemented or otherwise with MnTBAP at the concentration of 10 μg/ml. The transaminases were then assayed in the fluid at various times after removal of the organ. The addition of MnTBAP to a Belzer's fluid causes a reduction in the release of transaminases by the liver, indicating a reduction in hepatic cytolysis.

### EXAMPLE 5

### Comparison of the Activities of MnTBAP, of N-acetyl-L-cysteine, and of CuDIPS on Acute Hepatic Insufficiency Induced by Acetaminophen

CuDIPS [Cu(II)-(diisopropylsalicylate)2] is a reference CuZnSOD mimetic (MC KENZIE et al., Br. J. Pharmacol. 127, 1159-1164, 1999). The effect of N-acetyl-L-cysteine (NAC), of CuDIPS, and of MnTBAP on the survival of mice after intraperitoneal injection of 1000 mg/kg of acetaminophen (APAP) was compared.

The experimental protocol followed is the same as that described in the example above.

MnTBAP, NAC, or CuDIPS are administered in the form of a bolus, preventively, 2 hours before (P) or, curatively, 6 hours after (C) the acetaminophen.

Various groups of animals received the following treatments:

Group I: PBS

Group II: MnTBAP 10 mg/kg

Group III: NAC 300 mg/kg

Group IV: APAP 1000 mg/kg

Group V: APAP 1000 mg/kg; MnTBAP 10 mg/kg (P)

Group VI: APAP 1000 mg/kg; MnTBAP 20 mg/kg (P)

Group VII: APAP 1000 mg/kg; MnTBAP 10 mg/kg (C)

Group VIII: APAP 1000 mg/kg; MnTBAP 20 mg/kg (C)

Group IX: APAP 1000 mg/kg; MnTBAP 50 mg/kg (P) per os

Group X: APAP 1000 mg/kg; NAC 100 mg/kg (P)

Group XI: APAP 1000 mg/kg; NAC 200 mg/kg (P)

Group XII: APAP 1000 mg/kg; NAC 300 mg/kg (P)

Group XIII: APAP 1000 mg/kg; NAC 100 mg/kg (C)

Group XIV: APAP 1000 mg/kg; NAC 300 mg/kg (C)

Group XV: APAP 1000 mg/kg; CuDIPS 10 mg/kg (P)

The survival of the animals is monitored for 24 hours after administration of acetaminophen.

The results, expressed as a percentage of surviving animals, are illustrated by Table I below.

These results show that:

MnTBAP administered preventively increases the survival rate in a manner at least equal to NAC;

MnTBAP is active when it is administered by the oral route;

Unlike NAC, which is only active preventively, MnTBAP is active when it is administered curatively;

CuDIPS does not significantly increase the survival rate.

TABLE 1

| Time | 0 | 6 | 8 | 9 | 10 | 11 | 12 | 13 | 14 | 15 | 16 | 17 | 18 | 20 | 22 | 24 | No. |
|---|---|---|---|---|---|---|---|---|---|---|---|---|---|---|---|---|---|
| Group 1 | 100 | 100 | 100 | 100 | 100 | 100 | 100 | 100 | 100 | 100 | 100 | 100 | 100 | 100 | 100 | 100 | 16 |
| PBS | | | | | | | | | | | | | | | | | |
| Group II | 100 | 100 | 100 | 100 | 100 | 100 | 100 | 100 | 100 | 100 | 100 | 100 | 100 | 100 | 100 | 100 | 16 |
| MnTBAP 10 mg/kg | | | | | | | | | | | | | | | | | |
| Group III | 100 | 100 | 100 | 100 | 100 | 100 | 100 | 100 | 100 | 100 | 100 | 100 | 100 | 100 | 100 | 100 | 16 |
| NAC 300 mg/kg | | | | | | | | | | | | | | | | | |
| Group IV | 100 | 100 | 86.8 | 65.8 | 60.5 | 42.1 | 39.5 | 36.8 | 34.2 | 31.6 | 29 | 29 | 21 | 21 | 18.4 | 18.4 | 38 |
| APAP 1 g/kg | | | | | | | | | | | | | | | | | |
| Group V | 100 | 100 | 100 | 91.4 | 88.6 | 77.1 | 74.3 | 74.3 | 71.4 | 71.4 | 68.6 | 68.6 | 65.7 | 65.7 | 65.7 | 62.9 | 35 |
| APAP 1000-MnTBAP | | | | | | | | | | | | | | | | | |
| 10 mg/kg (P) | | | | | | | | | | | | | | | | | |
| Group VI | 100 | 100 | 100 | 100 | 100 | 100 | 100 | 100 | 100 | 90 | 90 | 90 | 90 | 90 | 90 | 90 | 10 |
| APAP 1000-MnTBAP | | | | | | | | | | | | | | | | | |
| 20 mg/kg (P) | | | | | | | | | | | | | | | | | |
| Group VII | 100 | 100 | 100 | 100 | 94 | 94 | 81 | 69 | 69 | 69 | 56 | 56 | 56 | 56 | 56 | 44 | 16 |
| APAP 1000-MnTBAP | | | | | | | | | | | | | | | | | |
| 10 mg/kg (C) | | | | | | | | | | | | | | | | | |
| Group VIII | 100 | 100 | 100 | 100 | 100 | 100 | 90 | 90 | 90 | 90 | 90 | 80 | 80 | 70 | 70 | 70 | 10 |
| APAP 1000-MnTBAP | | | | | | | | | | | | | | | | | |
| 20 mg/kg (C) | | | | | | | | | | | | | | | | | |
| Group IX | 100 | 100 | 88.8 | 88.8 | 88.8 | 88.8 | 88.8 | 88.8 | 88.8 | 88.8 | 88.8 | 77.7 | 66.6 | 66.6 | 66.6 | 66.6 | 9 |
| APAP 1000-MnTBAP | | | | | | | | | | | | | | | | | |
| 50 mg/kg (P) per | | | | | | | | | | | | | | | | | |
| os | | | | | | | | | | | | | | | | | |
| Group X | 100 | 100 | 100 | 100 | 100 | 100 | 100 | 100 | 90 | 80 | 60 | 60 | 50 | 40 | 40 | 40 | 10 |
| APAP 1000-NAC | | | | | | | | | | | | | | | | | |
| 100 (P) | | | | | | | | | | | | | | | | | |
| Group XI | 100 | 100 | 100 | 100 | 100 | 90 | 90 | 90 | 90 | 90 | 80 | 80 | 60 | 60 | 60 | 60 | 10 |
| APAP 1000-NAC | | | | | | | | | | | | | | | | | |
| 200 (P) | | | | | | | | | | | | | | | | | |
| Group XII | 100 | 100 | 100 | 90 | 90 | 90 | 90 | 90 | 90 | 90 | 90 | 90 | 80 | 80 | 80 | 80 | 10 |
| APAP 1000-NAC | | | | | | | | | | | | | | | | | |
| 300 (P) | | | | | | | | | | | | | | | | | |
| Group XIII | 100 | 100 | 90 | 80 | 60 | 50 | 50 | 40 | 30 | 20 | 20 | 20 | 10 | 10 | 10 | 10 | 10 |
| APAP 1000-NAC | | | | | | | | | | | | | | | | | |
| 100 (C) | | | | | | | | | | | | | | | | | |
| Group XIV | 100 | 100 | 100 | 90 | 80 | 80 | 60 | 40 | 40 | 40 | 30 | 30 | 30 | 20 | 20 | 20 | 10 |
| APAP 1000-NAC | | | | | | | | | | | | | | | | | |
| 300 (C) | | | | | | | | | | | | | | | | | |
| Group XV | 100 | 100 | 91.6 | 83.3 | 75 | 58.3 | 58.3 | 41.6 | 41.6 | 41.6 | 33.3 | 33.3 | 25 | 25 | 25 | 25 | 12 |
| APAP 1000-CuDIPS | | | | | | | | | | | | | | | | | |
| 10 mg/kg (P) | | | | | | | | | | | | | | | | | |

1. The use of a manganese superoxide dismutase (MnSOD) mimetic for producing a medicament intended for the preventive or curative treatment of hepatocellular insufficiency.

2. The use as claimed in claim 1, characterized in that said MnSOD mimetic possesses, in addition, a catalase activity and/or a glutathione peroxidase activity.

3. The use as claimed in either of claims 1 and 2, characterized in that said MnSOD mimetic is MnTBAP.

4. The use as claimed in any one of claims 1 to 3, characterized in that said MnSOD mimetic is used for producing a medicament intended for the preventive or curative treatment of hepatocellular insufficiency of toxic origin.

5. The use as claimed in claim 4, characterized in that said MnSOD mimetic is used for producing a medicament intended for the preventive or curative treatment of hepatocellular insufficiency induced by acetaminophen.

6. The use as claimed in any one of claims 1 to 3, characterized in that said MnSOD mimetic is used for producing a medicament intended for the preventive or curative treatment of hepatocellular insufficiency resulting from Fas-dependent apoptosis of the hepatocytes.

7. The use as claimed in claim 6, characterized in that said MnSOD mimetic is used for producing a medicament intended for the preventive or curative treatment of hepatocellular insufficiency of viral origin.

8. The use as claimed in any one of claims 1 to 4, characterized in that said MnSOD mimetic is used for producing a medicament intended for the preventive or curative treatment of hepatocellular insufficiency induced by alcohol.

9. The use as claimed in any one of claims 1 to 8, characterized in that said MnSOD mimetic is used for producing a medicament intended for the treatment of hepatocellular insufficiency manifesting itself in the form of fulminant hepatitis.

10. The use as claimed in any one of claims 1 to 9, characterized in that said MnSOD mimetic is used for producing a medicament intended for a preventive treatment.

11. The use as claimed in any one of claims 1 to 9, characterized in that said MnSOD mimetic is used for producing a medicament intended for a curative treatment.

12. The use as claimed in claim 10, characterized in that said medicament is formulated to allow the administration of an MnSOD mimetic dose of between 0.1 and 10 mg/kg/day.

13. The use as claimed in claim 11, characterized in that said medicament is formulated to allow the administration of an MnSOD mimetic dose of between 5 and 50 mg/kg/day.

14. The use of an MnSOD mimetic for protecting hepatic grafts during their preservation.
